(19) United States
(12) Patent Application Publication
(10) Pub. No.: US 2022/0189641 A1
(43) Pub. Date: Jun. 16, 2022

(54) Opioid Use Disorder Predictor

(71) Applicant: CERNER INNOVATION, INC., Kansas City, KS (US)

(72) Inventors: Alan Staples, Shawnee, KS (US); Vadim Khotilovich, Leawood, KS (US); Jennifer Martin, Moline, IL (US); Bennett Lovejoy, Gladstone, MO (US); Megan Barbre, Kansas City, KS (US); Lindsey Gay Jarrett, Kansas City, MO (US); Leslie Lindsey, Liberty, MO (US)

(21) Appl. No.: 17/123,520

(22) Filed: Dec. 16, 2020

(51) Int. Cl. G16H 50/70; G16H 50/30; G16H 50/20; G16H 20/10; G16H 40/20; G16H 10/60; G16H 10/40; G16H 10/20; G06Q 50/22; G16H 15/00; A61B 5/00; G09B 19/00

(52) U.S. Cl. CPC G16H 50/70; G09B 19/00; G16H 50/20; G16H 20/10; G16H 40/20; G16H 10/60; G16H 10/40; G16H 10/20; G06Q 50/22; G16H 15/00; A61B 5/7267; A61B 5/7275; A61B 5/4845; A61B 5/746; A61B 5/486; G16H 50/30

(57) ABSTRACT

Technologies are provided for leveraging machine learning to predict the likelihood of near-future OUD for patients presenting in an emergency department. A model is trained with data corresponding to one or more of: gender, age, prior opioid use disorder diagnosis, prior opioid use disorder events, prior opioids, prior emergency department encounters, prior inpatient encounters, other medications, drug screening tests, hepatitis C tests, tobacco use questionnaires, prior results from medical tests, social history questionnaires, or other diagnoses to predict opioid use disorder risk for a population of patients. Upon receiving information available at an emergency department triage for a patient, the trained model is utilized to predict the opioid use disorder risk for the patient over a predetermined period of time. The predicted opioid use disorder risk for the patient is provided within the patient chart over the predetermined period of time and may include details corresponding to risk factors specific to the patient and risk mitigation options.

### BACKGROUND

Opioid use disorder (OUD) is a chronic mental illness that often goes untreated. Moreover, OUD often goes undiagnosed. For example, a patient may have back pain and have either not seen a physician or the physician has not prescribed a medication. That patient may tell a friend who offers leftover pain medication (e.g., Percocet) the friend no longer needs. Even though the patient has never been opioids, they may be at risk for developing OUD. In this instance, the physician would never know the patient had used opioids unless the patient shares the information in a visit.

Even if the physician begins to see clues that the patient is experiencing OUD, the stigma of an OUD diagnosis may cause the physician to hesitate. In some cases, the physician may be concerned that if the patient is diagnosed with OUD, the patient may have difficulty getting insurance approval for certain procedures. For example, the patient may not be able to get approval for elective procedures where opioids are commonly prescribed for pain during recovery.

Conventional systems are unable to accurately predict OUD before the patient presents with symptoms of the disease. Rather, these systems rely on historical use and the like and are unable to predict future OUD. Unfortunately, the stigma with an OUD diagnosis described above may hinder the patient from receiving certain care, and worse, the patient may lose any opportunity to understand the risk of OUD and take preventative measures before it is too late.

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

Embodiments of the present invention relate to predicting the likelihood of near-future OUD. More particularly, embodiments of the present invention leverage machine learning to predict the likelihood of near-future OUD for patients presenting in an emergency department. To do so, a model is trained with data corresponding to one or more of: gender, age, prior opioid use disorder diagnosis, prior opioid use disorder events, prior opioids, prior emergency department encounters, prior inpatient encounters, other medications, or other diagnoses to predict opioid use disorder risk for a population of patients. Upon receiving information available at an emergency department (ED) triage for a patient, the trained model is utilized to predict the opioid use disorder risk for the patient over a predetermined period of time. The predicted opioid use disorder risk for the patient is provided within the patient chart over the predetermined period of time and may include details corresponding to risk factors specific to the patient, and one or more risk mitigation options.

### BRIEF DESCRIPTION OF THE DRAWINGS

The present invention is described in detail below with reference to the attached drawing figures, wherein:

FIG. 1 is a block diagram of an exemplary computing environment suitable for use in implementing the present disclosure;

FIG. 2 is a block diagram of an exemplary system for utilizing machine learning to predict the likelihood of near-future OUD for patients presenting in an emergency department, in accordance with an embodiment of the present disclosure;

FIG. 3 is a block diagram of an exemplary implementation of an opioid use disorder (OUD) engine, in accordance with some embodiments of the present disclosure;

FIGS. 4-8 depict illustrative screen displays, in accordance with embodiments of the present disclosure;

FIG. 9 is a flow diagram showing an exemplary method for utilizing machine learning to predict the likelihood of near-future OUD for patients presenting in an emergency department, in accordance with various embodiments of the present disclosure;

### DETAILED DESCRIPTION

The subject matter of the present invention is described with specificity herein to meet statutory requirements. However, the description itself is not intended to limit the scope of this patent. Rather, the inventors have contemplated that the claimed subject matter might also be embodied in other ways, to include different steps or combinations of steps similar to the ones described in this document, in conjunction with other present or future technologies. Moreover, although the terms “step” and/or “block” may be used herein to connote different components of methods employed, the terms should not be interpreted as implying any particular order among or between various steps herein disclosed unless and except when the order of individual steps is explicitly described.

As noted in the Background, conventional systems are unable to accurately predict OUD before the patient presents with symptoms of the disease. Rather, these systems rely on historical use and the like and are unable to predict future OUD. Unfortunately, the stigma with an OUD diagnosis described above may hinder the patient from receiving certain care, and worse, the patient may lose any opportunity to understand the risk of OUD and take preventative measures before it is too late.

Embodiments of the present invention relate to predicting the likelihood of near-future OUD. More particularly, embodiments of the present invention leverage machine learning to predict the likelihood of near-future OUD for patients presenting in an emergency department. To do so, a model is trained with data corresponding to one or more of: gender, age, prior opioid use disorder diagnosis, prior opioid use disorder events, prior opioids, prior emergency department encounters, prior inpatient encounters, other medications, or other diagnoses to predict opioid use disorder risk for a population of patients. Upon receiving information available at an ED triage for a patient, the trained model is utilized to predict the opioid use disorder risk for the patient over a predetermined period of time. The predicted opioid use disorder risk for the patient is provided within the patient chart over the predetermined period of time and may include details corresponding to risk factors specific to the patient, and one or more risk mitigation options.

In embodiments, a user interface presents the risk for a particular patient that may be stratified according to a high, medium, low, or unknown risk. Moreover, the user interface provides detailed information that may explain the factors that were considered by the model and contributed to the risk. Additionally, the user interface provides options that the clinician may utilize to help mitigate the risk. Together, the risk, the detailed information, and the mitigation options may enable a clinician to predict a patient that is going to experience OUD and potentially educate the patient on their particular risk so specific interventions and actions may be taken so the patient will not experience OUD in the future, or prepare the patient so any future OUD events are more likely to be survivable.

Accordingly, in one aspect, an embodiment of the present invention is directed to a computerized method. The method includes training a model to predict opioid use disorder risk for a population of patients. The model is trained with data corresponding to one or more of: gender, age, prior opioid use disorder diagnosis, prior opioid use disorder events, prior opioids, prior emergency department encounters, prior inpatient encounters, other medications, drug screening tests, hepatitis C tests, tobacco use questionnaires, prior results from medical tests, social history questionnaires, or other diagnoses. The method also includes receiving information available at an ED triage for a patient. The method further includes, in response to the receiving, utilizing the trained model to predict the opioid use disorder risk for the patient over a predetermined period of time. The method also includes providing, within the patient chart, the predicted opioid use disorder risk for the patient over the predetermined period of time. The predicted opioid use disorder risk includes details corresponding to risk factors specific to the patient, and one or more risk mitigation options.

In another aspect of the invention, an embodiment is directed to one or more computer storage media having computer-executable instructions embodied thereon that, when executed by a computer, causes the computer to perform operations. The operations include training a model to predict opioid use disorder risk for a population of patients. The model is trained with data corresponding to one or more of: gender, age, prior opioid use disorder diagnosis, prior opioid use disorder events, prior opioids, prior emergency department encounters, prior inpatient encounters, other medications, drug screening tests, hepatitis C tests, tobacco use questionnaires, prior results from medical tests, social history questionnaires, or other diagnoses. The operations also include receiving information available at an ED triage for a patient. The operations further include, in response to the receiving, utilizing the trained model to predict the opioid use disorder risk for the patient over a predetermined period of time. The operations also include providing, within the patient chart, the predicted opioid use disorder risk for the patient over the predetermined period of time.

In a further aspect, an embodiment is directed to a computerized system that includes one or more processors and a non-transitory computer storage medium storing computer-useable instructions that, when used by the one or more processors, cause the one or more processors to: train a model to predict opioid use disorder risk for a population of patients, the model trained with data corresponding to one or more of: gender, age, prior opioid use disorder diagnosis, prior opioid use disorder events, prior opioids, prior emergency department encounters, prior inpatient encounters, other medications, drug screening tests, hepatitis C tests, tobacco use questionnaires, prior results from medical tests, social history questionnaires, or other diagnoses; receive information available at an ED triage for a patient; in response to receiving the information, utilize the trained model to predict the opioid use disorder risk for the patient over a predetermined period of time; and provide, within the patient chart, the predicted opioid use disorder risk for the patient over the predetermined period of time, the predicted opioid use disorder risk including details corresponding to risk factors specific to the patient, and one or more risk mitigation options.

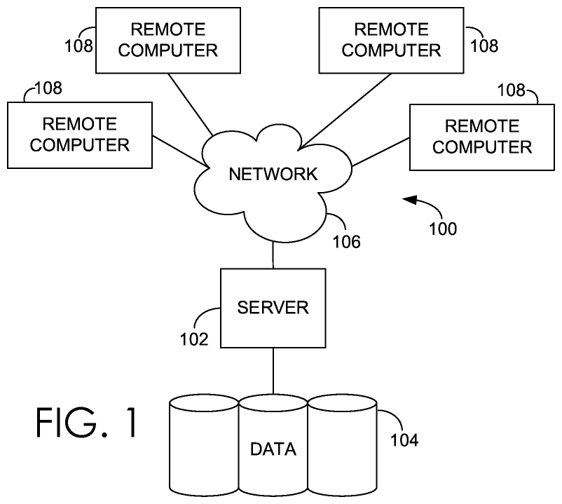

An exemplary computing environment suitable for use in implementing embodiments of the present invention is described below. FIG. 1 is an exemplary computing environment (e.g., medical-information computing-system environment) with which embodiments of the present invention may be implemented. The computing environment is illustrated and designated generally as reference numeral 100. The computing environment 100 is merely an example of one suitable computing environment and is not intended to suggest any limitation as to the scope of use or functionality of the invention. Neither should the computing environment 100 be interpreted as having any dependency or requirement relating to any single component or combination of components illustrated therein.

The present invention might be operational with numerous other purpose computing system environments or configurations. Examples of well-known computing systems, environments, and/or configurations that might be suitable for use with the present invention include personal computers, server computers, hand-held or laptop devices, multiprocessor systems, microprocessor-based systems, set top boxes, programmable consumer electronics, network PCs, minicomputers, mainframe computers, distributed computing environments that include any of the above-mentioned systems or devices, and the like.

The present invention might be described in the general context of computer-executable instructions, such as program modules, being executed by a computer. Exemplary program modules comprise routines, programs, objects, components, and data structures that perform particular tasks or implement particular abstract data types. The present invention might be practiced in distributed computing environments where tasks are performed by remote processing devices that are linked through a communications network. In a distributed computing environment, program modules might be located in association with local and/or remote computer storage media (e.g., memory storage devices).

With continued reference to FIG. 1, the computing environment 100 comprises a computing device in the form of a control server 102. Exemplary components of the control server 102 comprise a processing unit, internal system memory, and a suitable system bus for coupling various system components, including data store 104, with the control server 102. The system bus might be any of several types of bus structures, including a memory bus or memory controller, a peripheral bus, and a local bus, using any of a variety of bus architectures. Exemplary architectures comprise Industry Standard Architecture (ISA) bus, Micro Channel Architecture (MCA) bus, Enhanced ISA (EISA) bus, Video Electronic Standards Association (VESA) local bus, and Peripheral Component Interconnect (PCI) bus, also known as Mezzanine bus.

The control server 102 typically includes therein, or has access to, a variety of computer-readable media. Computer-readable media can be any available media that might be accessed by control server 102, and includes volatile and nonvolatile media, as well as, removable and nonremovable media. By way of example, and not limitation, computer-readable media may comprise computer storage media and communication media. Computer storage media includes both volatile and nonvolatile, removable and non-removable media implemented in any method or technology for storage of information such as computer-readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by control server 102. Communication media typically embodies computer-readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of any of the above should also be included within the scope of computer-readable media.

The control server 102 might operate in a computer network 106 using logical connections to one or more remote computers 108. Remote computers 108 might be located at a variety of locations in a medical or research environment, including clinical laboratories (e.g., molecular diagnostic laboratories), hospitals and other inpatient settings, ambulatory settings, medical billing and financial offices, hospital administration settings, home healthcare environments, clinicians' offices, Center for Disease Control, Centers for Medicare & Medicaid Services, World Health Organization, any governing body either foreign or domestic, Health Information Exchange, and any healthcare/government regulatory bodies not otherwise mentioned. Clinicians may comprise a treating physician or physicians; specialists such as intensivists, surgeons, radiologists, cardiologists, and oncologists; emergency medical technicians; physicians' assistants; nurse practitioners; nurses; nurses' aides; pharmacists; dieticians; microbiologists; laboratory experts; laboratory technologists; genetic counselors; researchers; students; and the like. The remote computers 108 might also be physically located in nontraditional medical care environments so that the entire healthcare community might be capable of integration on the network. The remote computers 108 might be personal computers, servers, routers, network PCs, peer devices, other common network nodes, or the like and might comprise some or all of the elements described above in relation to the control server 102. The devices can be personal digital assistants or other like devices.

Computer networks 106 comprise local area networks (LANs) and/or wide area networks (WANs). Such networking environments are commonplace in offices, enterprise-wide computer networks, intranets, and the Internet. When utilized in a WAN networking environment, the control server 102 might comprise a modem or other means for establishing communications over the WAN, such as the Internet. In a networking environment, program modules or portions thereof might be stored in association with the control server 102, the data store 104, or any of the remote computers 108. For example, various application programs may reside on the memory associated with any one or more of the remote computers 108. It will be appreciated by those of ordinary skill in the art that the network connections shown are exemplary and other means of establishing a communications link between the computers (e.g., control server 102 and remote computers 108) might be utilized.

In operation, an organization might enter commands and information into the control server 102 or convey the commands and information to the control server 102 via one or more of the remote computers 108 through input devices, such as a keyboard, a pointing device (commonly referred to as a mouse), a trackball, or a touch pad. Other input devices comprise microphones, satellite dishes, scanners, or the like. Commands and information might also be sent directly from a remote healthcare device to the control server 102. In addition to a monitor, the control server 102 and/or remote computers 108 might comprise other peripheral output devices, such as speakers and a printer.

Although many other internal components of the control server 102 and the remote computers 108 are not shown, such components and their interconnection are well known. Accordingly, additional details concerning the internal construction of the control server 102 and the remote computers 108 are not further disclosed herein.

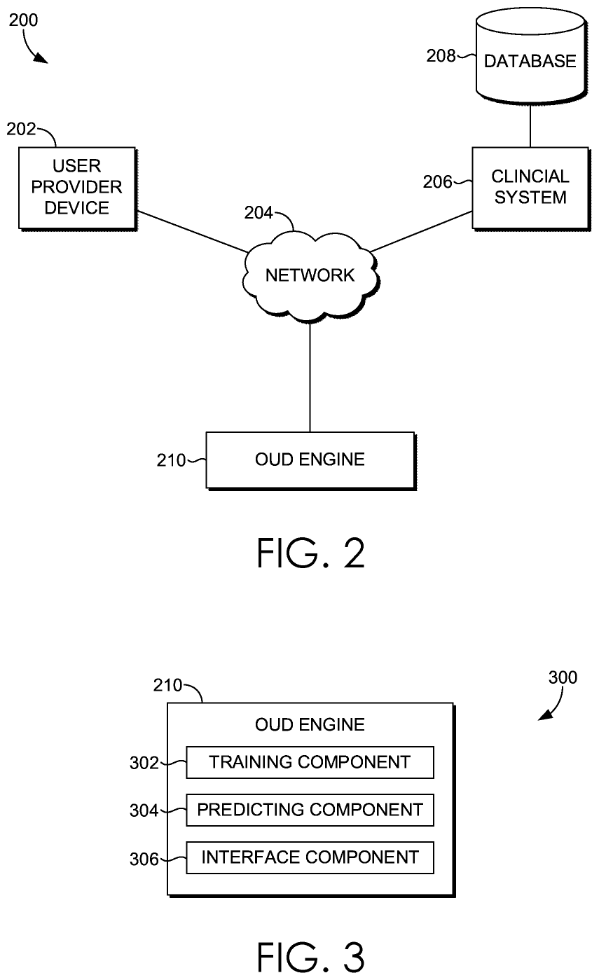

Turning now to FIG. 2, an exemplary OUD predictor system 200 is depicted suitable for use in implementing embodiments of the present invention. The OUD predictor system 200 is merely an example of one suitable computing system environment and is not intended to suggest any limitation as to the scope of use or functionality of embodiments of the present invention. Neither should the OUD predictor system 200 be interpreted as having any dependency or requirement related to any single module/component or combination of modules/components illustrated therein.

The OUD predictor system 200 includes user provider device 202, clinical system 206, database 208, and OUD engine 210, all in communication with one another via a network 204. The network 204 may include, without limitation, one or more secure local area networks (LANs) or wide area networks (WANs). The network 204 may be a secure network associated with a facility such as a healthcare facility. The secure network may require that a user log in and be authenticated in order to send and/or receive information over the network.

The components/modules illustrated in FIG. 2 are exemplary in nature and in number and should not be construed as limiting. Any number of components/modules may be employed to achieve the desired functionality within the scope of embodiments hereof. Further, components/modules may be located on any number of servers. By way of example only, OUD engine 210 might reside on a server, cluster of servers, or a computing device remote from one or more of the remaining components. Although illustrated as separate systems, the functionality provided by each of these components might be provided as a single component/module. The single unit depictions are meant for clarity, not to limit the scope of embodiments in any form.

Components of the OUD predictor system 200 may include a processing unit, internal system memory, and a suitable system bus for coupling various system components, including one or more data stores for storing information (e.g., files and metadata associated therewith). Components of the OUD predictor system 200 typically includes, or has access to, a variety of computer-readable media.

It should be understood that this and other arrangements described herein are set forth only as examples. Other arrangements and elements (e.g., machines, interfaces, functions, orders, and groupings of functions, etc.) can be used in addition to or instead of those shown, and some elements may be omitted altogether. Further, many of the elements described herein are functional entities that may be implemented as discrete or distributed components or in conjunction with other components/modules, and in any suitable combination and location. Various functions described herein as being performed by one or more entities may be carried out by hardware, firmware, and/or software. For instance, various functions may be carried out by a processor executing instructions stored in memory.

Clinical system 206 includes or has access to infrastructure that is capable of receiving and communicating information for use by, for example, OUD engine 210. The information received and communicated in association with clinical system 206 may comprise general information used by OUD engine 210. Clinical system 206 may receive data from user provider device 202 or other systems (e.g., disparate healthcare systems), which may include any number or type of medical devices that may be utilized to provide or measure patient care to a patient. In some embodiments, clinical system 206 may receive data from health information exchanges (“HIEs”), personal health records (“PHRs”), patient claims, and other health records associated with a patient.

Clinical system 206 includes or has access to infrastructure that is capable of storing electronic health records (EHRs), such as database 208 of patients associated with clinical system 206. EHRs may comprise records of treatment events, medication history, substance use history, demographic attributes, laboratory tests, time and date data, any other health related data, or any combination thereof for a plurality of patients. EHRs may also comprise electronic clinical documents such as images, clinical notes, orders, summaries, reports, analyses, or other types of electronic medical documentation relevant to a particular patient's condition and/or treatment. Electronic clinical documents contain various types of information relevant to the condition and/or treatment of a particular patient and can include information relating to, for example, patient identification information, images, alert history, culture results, physical examinations, vital signs, past medical histories, surgical histories, family histories, histories of present illnesses, current and past medications, allergies, symptoms, past orders, completed orders, pending orders, tasks, lab results, other test results, patient encounters and/or visits, immunizations, physician comments, nurse comments, other caretaker comments, and a host of other relevant clinical information.

In some embodiments, EHR 208 can include GC/MS results for THC, THC metabolites, opioids, opioid metabolites (e.g., the metabolites of heroin, opium, morphine, hydrocodone, oxycodone, and so forth), and other pharmaceuticals or metabolites thereof. Additionally, EHR 208 can include clinical notes, appointment notes, records of issued prescriptions, diagnoses, care plans, bloodwork, urinalysis, respiratory data, and treatment data for each patient of a healthcare institution or a plurality of healthcare institutions. Further, EHR 208 can include images, representations, or clinical documentation of burns, needle marks or unexplained abrasions, shakes/tremors, lack of personal hygiene, scars, unsubstantiated pains, requests for opioids or pain medication, etc. Additionally, in some embodiments, the EHR 208 can maintain one or more pharmaceutical formularies that identify the chemical composition of pharmaceuticals prescribed by, or available for prescription by, care providers. The formularies can further include keywords, potential for addiction information, dosage guidelines, generic and non-generic names, national drug codes (NDC), and any other data related to prescribeable pharmaceuticals. In some embodiments, a specific potential-for-addiction formulary can include a subset of pharmaceuticals with a known potential for abuse or addiction.

User provider device 202 may be any type of computing device used within a healthcare facility or as part of the claims processing process to receive, display, and send information to another user or system. User provider device 202 may be capable of communicating via the network with clinical system 206, database 208, or OUD engine 210. Such devices may include any type of mobile and portable devices including cellular telephones, personal digital assistants, tablet PCs, smart phones, and the like.

User provider device 202 is configured to display information to a clinician or user via a display. The information may include communications initiated by and/or received by OUD engine 210. Embodiments are not intended to be limited to visual display but rather may also include audio presentation, visual presentation, combined audio/visual presentation, and the like.

Generally, the OUD engine 210 is configured to leverage machine learning to predict the likelihood of near-future OUD for patients presenting in an emergency department. The OUD engine 210 facilitates acquisition and analysis of structured and unstructured data from one or more disparate data sources relevant to the OUD prediction. By way of example, the OUD engine 210 may include an application programing interface (API) library that includes specifications for routines, data structures, object classes, and variables that support the interaction of the OUD engine 210 architecture and the software framework of disparate data sources. Further, embodiments of the OUD engine 210 facilitate analysis of structured and unstructured data to generate a prediction of near-future OUD. As part of the analysis the OUD engine 210 can extract variables from the structured and unstructured data maintained by remote servers, local servers, or both. For example, the OUD engine 210 can invoke natural language processing algorithms to identify relevant variables in unstructured free text stored in a database maintained by one or more of the disparate data sources (e.g., a physician dictates in an emergency department note that the patient had to be resuscitated which may not be indicative or result in an OUD diagnosis but may be a risk factor that needs to be considered by the predictive model).

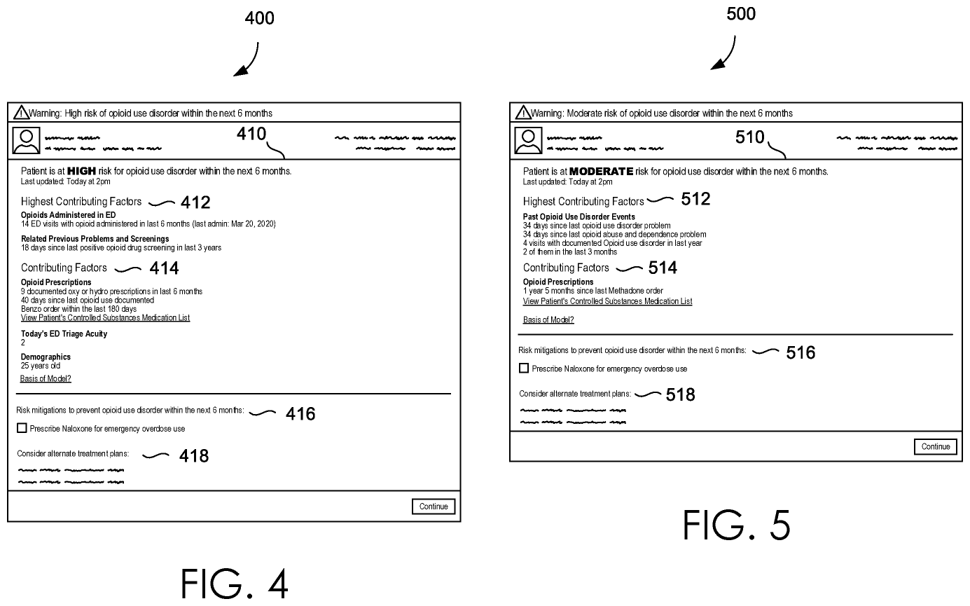

Referring now to FIG. 3, the OUD engine 210 includes several components. For example, the OUD engine 210 may include training component 302, a predicting component 304, and an interface component 306. Initially, the training component 302 generally facilitates generation and development of OUD prediction models. In some embodiments, multiple models can be generated by training component 302. For example, a first model can be generated for patients that have never been diagnosed with OUD and a second model can be generated for patients that have been previously diagnosed with OUD. As can be appreciated, additional models can be generated based on other factors such as patient demographics, family history, and the like.

Continuing, training component 302 uses historical records with known OUD statuses and machine learning techniques to generate and develop OUD prediction models in some embodiments. For example, a cohort of historical subjects in an electronic health records system with known OUD can be identified. Known OUD statuses may be determined in a variety of ways. In some embodiments, for example, training component 302 may identify a plurality of subjects with documented OUD treatment events (such as admission to an OUD recovery program, treatment for OUD, self-report, and similar events) or documented OUD events contained in the subject's electronic medical record. In an embodiment, training component 302 identifies subjects by ICD-9 and ICD-10-CM diagnoses with a range indicative of OUD events. Training component 302 then compiles the identified subjects into one or more cohorts. For example, training component 302 can compile a cohort including all identified subjects, a second cohort including subjects with documented OUD events, a third cohort including subjects with no history of OUD events, and so on.

In some embodiments, training component 302 retrieves historical values for treatment events, medication history, OUD history, physiologic values, demographic attributes, laboratory tests, or other features that are germane to treatment efficacy corresponding to each cohort subject. The historical values can be held as unstructured data, assembled into a temporal order, or both. In some embodiments, OUD engine further de-identifies/anonymizes the subjects included in the cohort. Training component 302 can use support vector machine (SVM), Naïve Bayes, boosted and bagged decision trees, k-nearest neighbor, discriminant analysis, logistic regression, supervised learning, or any combination thereof to build a prediction model, identify input variables, and identify pre-model condition algorithms for each cohort. Further, training component 302 can further identify input variables for the prediction model that reduce the likelihood of OUD. Said differently, some embodiments of training component 302 determine the weights or relationships of the historical data that indicate the likelihood of near-future OUD and the historical data that mitigates (or reduces) potential OUD.

OUD engine 210 generally conditions or otherwise prepares data based on inputs for prediction models generated by training component 302. OUD engine 210 includes natural language processing services (not shown) such as Discern nCode™ developed by Cerner Corporation, or similar services. Further, OUD engine 210 can load and execute operations or functions identified by the training component 302 for pre-model conditioning based on the information retrieved by training component 302. Additionally, OUD engine 210 includes a data store, which in some embodiments includes data for a target patient (or information for multiple patients), including raw data sets or input variables; variables associated with patient recommendations; recommendation knowledge base; recommendation rules; recommendations; recommendation update statistics; an operational data store, which stores events, frequent itemsets (such as “X often happens with Y”, for example), and item sets index information; association rulebases; agent libraries, solvers and solver libraries, for example. It is contemplated that the term data includes any information that can be stored in a computer-storage device or system, such as user-derived data, computer usable instructions, software applications, or other information.

Next, predicting component 304 generally, comprises the services or routines for forecasting likelihood of near-future OUD, which may be developed according to the method described in connection to training component 302. For example, predicting component 304 performs statistical software operations, and can include statistical calculation packages such as, in one embodiment, the R system (the R-project for Statistical Computing, which supports R-packages or modules tailored for specific statistical operations, and which is accessible through the Comprehensive R Archive Network (CRAN) at http://cran.r-project.org) or similar services.

Finally, interface component 306 generally facilitates access to the output of predicting component 304 associated with a target patient. The interface component 306 can store the output (e.g., the OUD risk, details corresponding to risk factors specific to the patient, and risk mitigation options) in the target patient's EHR, generate an alert visible when an authorized user accesses the target patient's EHR, generate and present an automatic alert coexistent with presentation of the OUD risk, or any combination thereof.

In an embodiment, OUD engine 210 includes the services or routines, which may be embodied as one or more software agents or routines. Some embodiments of OUD engine 210 may further use Apache Hadoop and Hbase framework (not shown), or similar frameworks operable for providing a distributed file system, and which in some embodiments facilitate provide access to cloud-based services such as those provided by Cerner Healthe Intent®. Additionally, some embodiments of OUD engine 210 may further comprise one or more stream processing service(s) (not shown). For example, such stream processing service(s) may be embodied using IBM InfoSphere stream processing platform, Twitter Storm stream processing, Ptolemy or Kepler stream processing software, or similar complex event processing (CEP) platforms, frameworks, or services, which may include multiple such stream processing services (in parallel, serially, or operating independently). Some embodiments of the invention also may be used in conjunction with Cerner Millennium®, Cerner CareAware® (including CareAware iBus®), Cerner CareCompass®, or similar products and services.

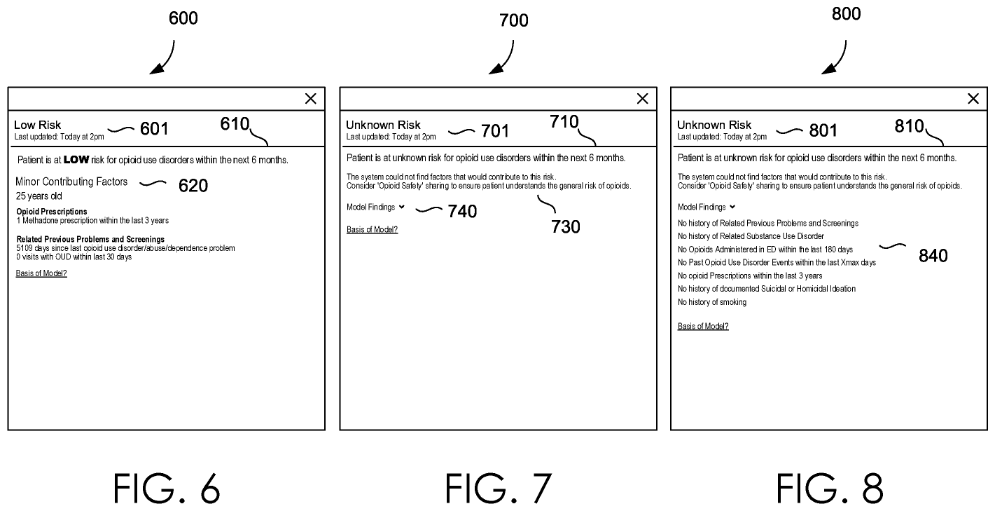

With reference to FIGS. 4-8, illustrative screen displays of embodiments of the present invention are shown. It is understood that each of the illustrative screen displays are connected logically, such that they comprise a user interface designed for providing information to the clinician on the elements in the chart that led to the risk displayed. The screen displays may appear in any order and with any number of screen displays, without regard to whether the screen display is described or depicted herein. The screen displays provide tools that utilize machine learning to predict the likelihood of near-future OUD for patients presenting in an emergency department, in accordance with embodiments of the present invention.

As shown in FIGS. 4-8, alert interfaces 400-800 include details interfaces 410, 510, 610, 710, 810. In particular, alert interface 400 illustrates a high risk alert 401 indicating that the patient has a high risk of opioid use disorder within the next six months. The details interface 410 further provides the highest contributing factors 412, other contributing factors 414, risk mitigations 416, and alternate treatment plans 418.

Turning to alert interface 500, a moderate risk alert 501 indicates the patient has a moderate risk of opioid use disorder within the next six months. Similar to details interface 410 of FIG. 4, details interface 510 provides the highest contributing factors 512, other contributing factors 514, risk mitigations 516, and alternate treatment plans 518.

In alert interface 600, a low risk alert 601 is provided. The low risk alert 601 indicates the patient has a low risk of opioid use disorder within the next six months. As shown, the alert interface includes details interface 610. In this example details interface 610, minor contributing factors 620 are provided. Although some minor contributing factors 620 may be identified, the OUD system may learn or determine that these minor contributing factors 620 are not predictive or not as predictive as other contributing factors or the highest contributing factors for developing OUD as illustrated in FIGS. 4 and 5.

Next, alert interface 700 provides an unknown risk alert 701. The unknown risk alert 701 may provide additional information 730 indicating that the OUD system was unable to identify any factors that contribute to OUD risk. Alert interface 700 may also provide model findings 740. Turning to alert interface 800, a clinician has expanded the model findings 840. Model findings 840 may provide support for why the OUD system was unable to identify any factors that contribute to OUD risk. For example, model findings 840 may indicate the patient has no history of related previous problems and screenings. In another example, model findings 840 may indicate the patient has no history of related substance use disorder. Model findings 840 may indicate the patient has not had opioids administered in an emergency department within the last predetermined number of days. Or, model findings 840 may indicate the patient has not had past opioid use disorder events within the last predetermined number of days. In yet another example, model findings may indicate the patient has not had any opioid prescriptions within the last predetermined number of years. Model findings 840 may further indicate the patient has no history of documented suicidal or homicidal ideation. Model findings 840 may also indicate the patient has no history of smoking.

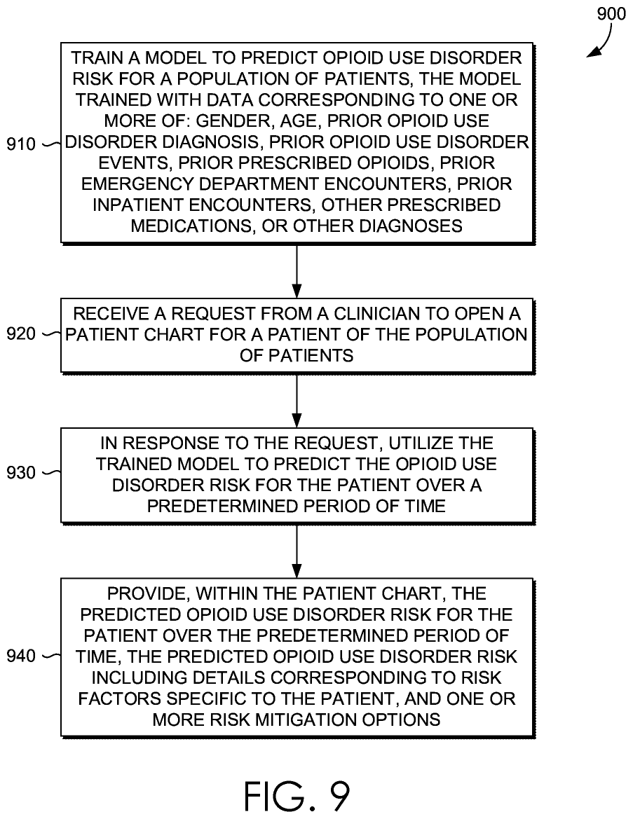

Referring to FIG. 9, a flow diagram is provided illustrating a method 900 for utilizing machine learning to predict the likelihood of near-future OUD for patients presenting in an emergency department, in accordance with various embodiments of the present disclosure, in accordance with various embodiments of the present disclosure. Method 900 may be performed by any computing device (such as computing device described with respect to FIG. 1) with access to an OUD predictor system (such as the one described with respect to FIG. 2) or by one or more components of the OUD predictor system (such as the OUD engine described with respect to FIGS. 2 and 3).

Initially, as shown at step 910, a model is trained to predict opioid use disorder risk for a population of patients. The model is trained with data corresponding to one or more of: gender, age, prior opioid use disorder diagnosis, prior opioid use disorder events, prior opioids, prior emergency department encounters, prior inpatient encounters, other medications, drug screening tests, hepatitis C tests, tobacco use questionnaires, prior results from medical tests, social history questionnaires, or other diagnoses.

In embodiments, two different models are trained. A first model may be trained for a portion of the population of patients that has been previously diagnosed with opioid use disorder. A second model may be trained for a portion of the population of patients that has not been previously diagnosed with opioid use disorder.

At step 920, information available at an emergency department triage for a patient is received. The information may include one or more of: gender, age, prior opioid use disorder diagnosis, prior opioid use disorder events, prior opioids, prior emergency department encounters, prior inpatient encounters, other medications, drug screening tests, hepatitis C tests, tobacco use questionnaires, prior results from medical tests, social history questionnaires, or other diagnoses. In response to receiving the information, at step 930, the trained model is utilized to predict the opioid use disorder risk for the patient over a predetermined period of time. For example, during the triage, historical data (such as from one or more electronic health records) for the patient may be automatically communicated to the trained model. The trained model utilizes the historical data to predict the opioid use disorder risk for the patient over the predetermined period of time. Additionally or alternatively, triage assessment information for the patient may be received. The triage assessment information for the patient is communicated to the trained model. The trained model utilizes the triage assessment information to predict the opioid use disorder risk for the patient over the predetermined period of time. As can be appreciated, such triage assessment information may be particularly valuable where no historical data is available.

At step 940, the predicted opioid use disorder risk for the patient over the predetermined period of time is provided within the patient chart. Upon determining the opioid use disorder risk for the patient exceeds on or more thresholds, an alert interface may be provided within the patient chart. Upon receiving an interaction with the alert interface within the patient chart, the predicted opioid use disorder risk and details may be provided in a detail interface within the patient chart.

For example, the details interface may include details corresponding to risk factors specific to the patient, and one or more risk mitigation options. In embodiments, the one or more risk mitigation options comprise one or more of: providing opioid safety education to the patient, providing education on alternate pain management methods to the patient, conducting an opioid use disorder screening, initiating screening, brief intervention, and referral to treatment (SBIRT) assessment, providing medication-assisted treatment (MAT), providing an order for a social worker, notifying a primary care physician of the patient, conducting a urine drug screening, prescribing or provisioning Naloxone, providing a referral for treatment, or following-up to confirm the patient attended a referral appointment. The system is not limited to the mitigations described above.

As can be understood, the present invention provides systems, methods, and user interfaces for utilizing machine learning to predict the likelihood of near-future OUD for patients presenting in an emergency department. The present invention has been described in relation to particular embodiments, which are intended in all respects to be illustrative rather than restrictive. Alternative embodiments will become apparent to those of ordinary skill in the art to which the present invention pertains without departing from its scope.

From the foregoing, it will be seen that this invention is one well adapted to attain all the ends and objects set forth above, together with other advantages which are obvious and inherent to the system and method. It will be understood that certain features and subcombinations are of utility and may be employed without reference to other features and subcombinations. This is contemplated and within the scope of the claims.

We claim:

1. A method comprising:
training a model to predict opioid use disorder risk for a population of patients, the model trained with data corresponding to one or more of: gender, age, prior opioid use disorder diagnosis, prior opioid use disorder events, prior opioids, prior emergency department encounters, prior inpatient encounters, other medications, drug screening tests, hepatitis C tests, tobacco use questionnaires, prior results from medical tests, social history questionnaires, or other diagnoses;
receiving information available at an emergency department triage for a patient;
in response to the receiving, utilizing the trained model to predict the opioid use disorder risk for the patient over a predetermined period of time; and
providing, within the patient chart, the predicted opioid use disorder risk for the patient over the predetermined period of time, the predicted opioid use disorder risk including details corresponding to risk factors specific to the patient, and one or more risk mitigation options.

2. The method of claim 1, wherein the trained model is trained for a portion of the population of patients that has been previously diagnosed with opioid use disorder.

3. The method of claim 1 wherein the trained model is trained for a portion of the population of patients that has not been previously diagnosed with opioid use disorder.

4. The method of claim 1, further comprising communicating historical data for the patient to the trained model, the trained model utilizing the historical data to predict the opioid use disorder risk for the patient over the predetermined period of time.

5. The method of claim 1, wherein the one or more risk mitigation options comprise one or more of: providing opioid safety education to the patient, providing education on alternate pain management methods to the patient, conducting an opioid use disorder screening, initiating screening, brief intervention, and referral to treatment (SBIRT) assessment, providing medication-assisted treatment (MAT), providing an order for a social worker, notifying a primary care physician of the patient, conducting a urine drug screening, prescribing or provisioning Naloxone, providing a referral for treatment, or following-up to confirm the patient attended a referral appointment.

6. The method of claim 1, further comprising communicating the information available at the triage assessment for the patient to the trained model, the trained model utilizing the information available at the triage assessment to predict the opioid use disorder risk for the patient over the predetermined period of time.

7. The method of claim 1, further comprising determining the opioid use disorder risk for the patient exceed one or more thresholds.

8. The method of claim 7, further comprising, based on the determining the opioid use disorder risk for the patient exceed the one or more thresholds, providing an alert interface within the patient chart.

9. The method of claim 8, further comprising, upon receiving an interaction with the alert interface within the patient chart, providing the predicted opioid use disorder risk and the details in a detail interface within the patient chart.

10. One or more computer storage media having computer-executable instructions embodied thereon that, when executed by a computer, causes the computer to perform operations comprising:
training a model to predict opioid use disorder risk for a population of patients, the model trained with data corresponding to one or more of: gender, age, prior opioid use disorder diagnosis, prior opioid use disorder events, prior opioids, prior emergency department encounters, prior inpatient encounters, other medications, drug screening tests, hepatitis C tests, tobacco use questionnaires, prior results from medical tests, social history questionnaires, or other diagnoses;
receiving information available at an emergency department triage for a patient;
in response to the receiving, utilizing the trained model to predict the opioid use disorder risk for the patient over a predetermined period of time; and
providing, within the patient chart, the predicted opioid use disorder risk for the patient over the predetermined period of time.

11. The media of claim 10, further comprising communicating historical data for the patient to the trained model, the trained model utilizing the historical data to predict the opioid use disorder risk for the patient over the predetermined period of time.

12. The media of claim 10, further comprising communicating the triage assessment information for the patient to the trained model, the trained model utilizing the triage assessment information to predict the opioid use disorder risk for the patient over the predetermined period of time.

13. The media of claim 10, further comprising:
determining the opioid use disorder risk for the patient exceed one or more thresholds; and
based on the determining the opioid use disorder risk for the patient exceed the one or more thresholds, providing an alert interface within the patient chart.

14. The media of claim 13, further comprising, upon receiving an interaction with the alert interface within the patient chart, providing the predicted opioid use disorder risk and the details in a detail interface within the patient chart.

15. The media of claim 10, wherein the trained model is trained for a portion of the population of patients that has been previously diagnosed with opioid use disorder and the patient has been previously diagnosed with opioid use disorder.

16. The media of claim 10, wherein the trained model is trained for a portion of the population of patients that has not been previously diagnosed with opioid use disorder and the patient has not been previously diagnosed with opioid use disorder.

17. The media of claim 10, wherein the predicted opioid use disorder risk include details corresponding to risk factors specific to the patient, and one or more risk mitigation options.

18. The media of claim 17, wherein the one or more risk mitigation options comprise one or more of: providing opioid safety education to the patient, providing education on alternate pain management methods to the patient, conducting an opioid use disorder screening, initiating screening, brief intervention, and referral to treatment (SBIRT) assessment, providing medication-assisted treatment (MAT), providing an order for a social worker, notifying a primary care physician of the patient, conducting a urine drug screening, prescribing or provisioning Naloxone, providing a referral for treatment, or following-up to confirm the patient attended a referral appointment.

19. A system comprising:
one or more processors; and
a non-transitory computer storage media storing computer-useable instructions that, when used by the one or more processors, cause the one or more processors to:
train a model to predict opioid use disorder risk for a population of patients, the model trained with data corresponding to one or more of: gender, age, prior opioid use disorder diagnosis, prior opioid use disorder events, prior opioids, prior emergency department encounters, prior inpatient encounters, other medications, drug screening tests, hepatitis C tests, tobacco use questionnaires, prior results from medical tests, social history questionnaires, or other diagnoses;
receive information available at an emergency department triage for a patient;
in response to the request, utilize the trained model to predict the opioid use disorder risk for the patient over a predetermined period of time; and
provide, within the patient chart, the predicted opioid use disorder risk for the patient over the predetermined period of time, the predicted opioid use disorder risk including details corresponding to risk factors specific to the patient, and one or more risk mitigation options.

20. The system of claim 19, further comprising upon receiving an interaction with the alert interface within the patient chart, providing the predicted opioid use disorder risk and the details in a detail interface within the patient chart.
